(19) United States
(12) Patent Application Publication
(10) Pub. No.: US 2026/0250244 A1
(43) Pub. Date: Aug. 27, 2026

(54) New Process for the Preparation of Fluoropyrrolidine

(71) Applicant: Hoffmann-La Roche Inc., Little Falls, NJ (US)

(72) Inventors: Pascal Stephan Engl, Dietikon (CH); Stefan Hildbrand, Gelterkinden (CH); Christian Steffen Moessner, Lörrach (DE); Markus Stoeckli, Dottikon (CH); Alfred Stutz, Dottikon (CH)

(73) Assignee: Hoffmann-La Roche Inc., Little Falls, NJ (US)

(21) Appl. No.: 19/652,903

(22) Filed: Apr. 20, 2026

Related U.S. Application Data
(63) Continuation of application No. PCT/EP2024/079613, filed on Oct. 21, 2024.

(30) Foreign Application Priority Data
Oct. 23, 2023 (EP) 23205203.5

(51) Int. Cl. C07D 207/10; C07D 207/12

(52) U.S. Cl. CPC C07D 207/10; C07D 207/12; C07B 2200/07; C07B 2200/13

(57) ABSTRACT

The present invention relates to a new process for the preparation of (3R)-3-fluoropyrrolidine hydrochloride or an acceptable salts thereof via a N-Boc-(3S)-3-hydroxypyrrolidine intermediate. The process is suitable for the synthesis of N-Boc-(3S)-3-hydroxypyrrolidine at technical scale in high, not less than 99.85%-a/a optical purity and a purity of not less than 99.8%-a/a. The high quality N-Boc-(3S)-3-hydroxypyrrolidine can be converted to (3R)-3-fluoropyrrolidine hydrochloride exhibiting an optical purity of not less than 99.95%-a/a and a purity of not less than 99.5%-a/a thus making it a suitable intermediate for manufacturing of pharmaceutical active compounds.

The present invention relates in particular to a new process for the preparation of high quality and purity (3R)-3-fluoropyrrolidine hydrochloride salt of formula (I),

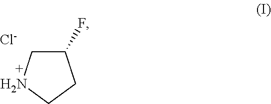

as well as intermediates thereof, both of which are useful building blocks for the manufacturing of pharmaceutical active compounds such as e.g. (3R)—N-[2-cyano-4-fluoro-3-(3-methyl-4-oxo-quinazolin-6-yl)oxy-phenyl]-3-fluoro-pyrrolidine-1-sulfonamide.

(3R)—N-[2-cyano-4-fluoro-3-(3-methyl-4-oxo-quinazolin-6-yl)oxy-phenyl]-3-fluoro-pyrrolidine-1-sulfonamide is a paradox breaking BRAF inhibitor for use in the treatment of patients having cancer with activating BRAF mutations. A synthetic route to obtain (3R)—N-[2-cyano-4-fluoro-3-(3-methyl-4-oxo-quinazolin-6-yl)oxy-phenyl]-3-fluoro-pyrrolidine-1-sulfonamide has already been disclosed in WO2021116050A1. Step 3 of this process uses (3R)-3-fluoropyrrolidine hydrochloride as a building block for synthesizing the building block (3R)-3-fluoropyrrolidine-1-sulfonamide, which can then be further processed to yield (3R)—N-[2-cyano-4-fluoro-3-(3-methyl-4-oxo-quinazolin-6-yl)oxy-phenyl]-3-fluoro-pyrrolidine-1-sulfonamide.

The purity of an active pharmaceutical ingredient is crucial to provide simultaneously an efficient and safe treatment to patients. In the present case, the optical purity of the (3R)-3-fluoropyrrolidine hydrochloride starting material may have a direct impact on the optical purity of the final product. In order for a safe and efficient long-term administration of (3R)—N-[2-cyano-4-fluoro-3-(3-methyl-4-oxo-quinazolin-6-yl)oxy-phenyl]-3-fluoro-pyrrolidine-1-sulfonamide, it is therefore crucial to develop a process that provides (3R)-3-fluoropyrrolidine hydrochloride in high yields and purity (not less than 99.5%-a/a, preferentially not less than 99.8%-a/a; not more of 0.05% of any individual impurity), can be used on technical scale, and delivers the required favourable optical purity with not more than 0.05%-a/a of the wrong enantiomer (optical purity not less than 99.95%-a/a). (3R)-3-fluoropyrrolidine hydrochloride is commercially available with a specified purity of not less than 98.0%-a/a and optical purity of not less than 99.85%-a/a and is thus not suitable for being used in the manufacture of (3R)—N-[2-cyano-4-fluoro-3-(3-methyl-4-oxo-quinazolin-6-yl)oxy-phenyl]-3-fluoro-pyrrolidine-1-sulfonamide.

The known methods of producing (3R)-3-fluoropyrrolidine hydrochloride have the disadvantage of favouring racemization of the chiral center to variable extent. Additionally, competing side reaction (such as HF or H₂O elimination, deoxychlorination due to Cl contamination, as well as hydrolysis and solvolysis) impact the selectivity and the overall purity of the final product. (3S)-3-Hydroxypyrrolidine or (3S)-3-hydroxypyrrolidine hydrochloride are suitable starting materials to produce (3R)-3-fluoropyrrolidine hydrochloride. However, commercially available (3S)-3-hydroxypyrrolidine and (3R)-3-fluoropyrrolidine hydrochloride are usually only specified with a purity of not less than 98.0%-a/a and an optical purity of not less than 99.0%-a/a. It has previously been described that chiral (3S)-3-hydroxypyrrolidine and derivatives thereof can be obtained from (S)-4-chloro-3-hydroxy-butanenitrile via self-cyclization in WO2007024113, however this procedure required protection of the hydroxyl group and therefore presents the disadvantage that additional steps are required thus increasing the time and cost of such a process when compared to the present process that instead uses direct hydrogenation without any protection group. In order to produce (3R)-3-fluoropyrrolidine hydrochloride from (3S)-3-hydroxypyrrolidine hydrochloride or the salt free analogue thereof, an O-activation is typically required, which will further also require transient N-protection with a suitable protection group prior to deoxyfluorination in view of the high chemical reactivity of the primary amine of the (3S)-3-hydroxypyrrolidine. The inventors of the present invention have developed a new procedure, wherein for protection of the primary amine the Boc protection group was chosen out of (a) convenience with respect to removal, and (b) good retention of optical purity during deoxyfluorination. One requirement to achieve high purity and optical purity, as well as high yields of (3R)-3-fluoropyrrolidine is therefore to obtain high quality (purity of not less than 99.8%-a/a and optical purity of not less than 99.85%-a/a) of the intermediate N-Boc-(3S)-3-hydroxypyrrolidine, as some optical purity will be typically lost during the deoxyfluorination process. The lower purity one obtains, the more crystallization cycles need to be conducted to achieve the required purity of not less than 99.5%-a/a and optical purity of not less than 99.95%-a/a, which will then result in loss of yield.

A further increase in purity and enantiomeric enrichment of commercial (3R)-3-fluoropyrrolidine hydrochloride requires multiple crystallizations with loss of yield and consequently higher costs associated with the process. In particular the impurities obtained from competing deoxychlorination and hydrolysis following O-activation exhibit a limited purge during crystallization and therefore represent critical impurities that need to be controlled during the manufacturing of (3R)-3-fluoropyrrolidine hydrochloride.

It was found that the objective of providing a process which is applicable on large scale and provides the required purity and optical purity of (3R)-3-fluoropyrrolidine hydrochloride could be reached with the new process of the present invention as described herein. The present invention relates to such a process wherein the product could be obtained via hydrogenation of (S)-4-chloro-3-hydroxy-butanenitrile without the need for prior 0-protection followed by isolation of an intermediate N-Boc-(3S)-3-hydroxypyrrolidine in high purity of not less than 99.8%-a/a and optical purity of not let than 99.85%-a/a, when using the nitrile starting material with an optical purity of not less than 99.3%-a/a. The high quality N-Boc-(3S)-3-hydroxypyrrolidine was then activated and deoxyfluorination followed by not more than two re-crystallizations provided the (3R)-3-fluoropyrrolidine hydrochloride product in the desired quality and purity.

The term “acceptable salt” refers to those salts of the compound as indicated, which retain the properties of the free bases, the free acids or the specifically disclosed salt form, which are not pharmaceutically or otherwise undesirable. In particular, it is to be understood that the term “acceptable salt” as used therein encompasses those salts that provide good crystallization, impurity rejection and/or are stable salts. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. The compound of formula (I) is a hydrochloride salt and even though a (3R)-3-fluoropyrrolidine:hydrochloride stoichiometry of about 1:1 appears favourable, the invention is not regarded to be limited in view of any deviating stoichiometry between the (3R)-3-fluoropyrrolidine and the hydrochloride.

An acceptable salt of the compound of formula (I) is to be understood as encompassing also acceptable salts of the compound of formula wherein the hydrochloride is replaced by a suitable alternative salt. In particular the compound (Ia), namely the mesylate salt, is intended to be included as an acceptable salt of the compound of formula (I):

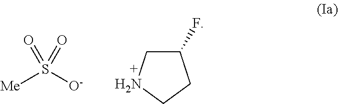 [SMILES: CS(=O)(=O)[O-] ; F[C@@H]1CCNC1]

The term “pump hydrogenation” refers to a dosage of a starting material solution over a defined duration to a previously prepared catalyst suspension (eventually with additives, if necessary) in the autoclave, which has already been brought to the required reaction temperature and hydrogen pressure. The starting material gets subsequently reduced immediately when it gets pumped (dosed) to the catalyst suspension in the autoclave.

The term “batch process” refers to a reaction process, where all starting materials including solvents and catalysts are present in a production vessel at reaction start.

If one of the starting materials, intermediates or final products as described herein contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, New York) can be introduced before the critical step applying methods well known in the art. Such protecting groups can be removed at a later stage of the synthesis using standard methods described in the literature. Examples of protecting groups are for instance tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compounds of formulas (C1), (D1), (E1), (C1′) and (D1′) all comprise a “Boc” protective group as described above. In principle, “Cbz” protecting group would be a suitable alternative to the use of “Boc”. The meaning of the “Boc” group is generally known to the person skilled in the art, but is nevertheless illustrated below exemplary in the compound of formula (C1):

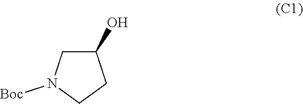 [SMILES: CC(C)(C)OC(=O)N1CC[C@H](O)C1]

is a abbreviated representation of

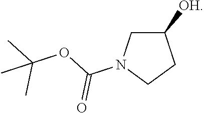

While the present invention has been described with reference to the specific embodiments thereof, it should be understood by those skilled in the art that various changes can be made and equivalents can be substituted without departing from the true spirit and scope of the invention. In addition, many modifications can be made to adapt a particular situation, material, composition of matter, process, process step or steps, to the objective spirit and scope of the present invention. All such modifications are intended to be within the scope of the claims appended hereto. All separate embodiments can be combined.

Specifically numbered embodiments of the invention are:
1. A process for the preparation of a compound of formula (I),

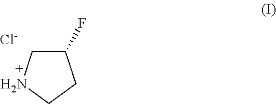

or an acceptable salt thereof, comprising the following steps:
(a) the reaction of a compound of formula (C1)

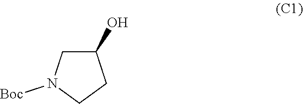 [SMILES: CC(C)(C)OC(=O)N1CC[C@H](O)C1]

with a compound (C2) selected from

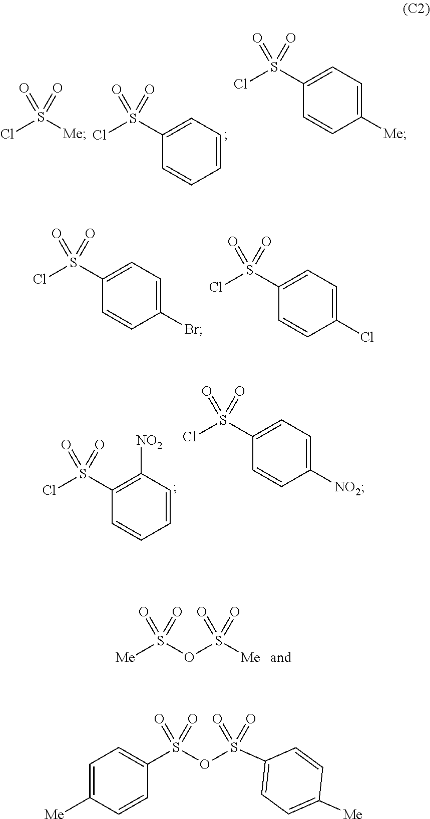

in presence of a suitable base (ii) and a suitable solvent (ii), to arrive at a compound of formula (D1)

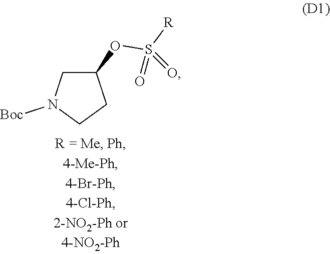

(b) the reaction of the compound of formula (D1) with a suitable fluorination agent in presence of a suitable solvent (iii) and optionally a suitable base (v), to arrive at a compound of formula (E1)

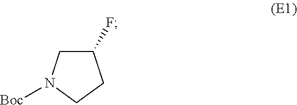 [SMILES: CC(C)(C)OC(=O)N1CC[C@@H](F)C1]

and
   (c) the reaction of the compound of formula (E1) with a suitable acid (i), in presence of a suitable solvent (iv), to arrive at a compound of formula (I) or an acceptable salt thereof.
2. A process for the preparation of a compound of formula (I),

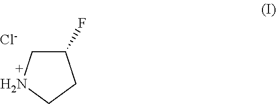

or an acceptable salt thereof, comprising the following steps:
(a) the reaction of a compound of formula (B1) or (B1′)

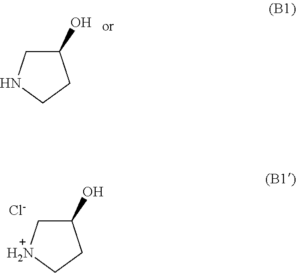 [SMILES: O[C@H]1CCNC1 ; [Cl-] ; O[C@H]1CC[NH2+]C1]

with a compound of formula (B2)

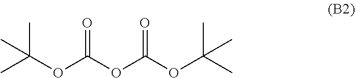

in presence of a suitable solvent (i) and optionally in presence of a suitable base (i), to arrive at a compound of formula (C1′)

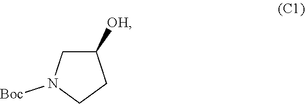 [SMILES: CC(C)(C)OC(=O)N1CC[C@H](O)C1]

(b) the reaction of the compound of formula (C1) with a compound of formula (C2′)

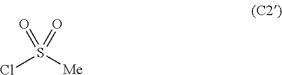

in presence of a suitable base (ii) and a suitable solvent (ii), to arrive at a compound of formula (D1′)

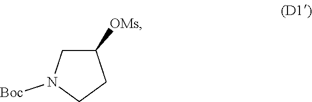 [SMILES: CC(C)(C)OC(=O)N1CC[C@H](OS(C)(=O)=O)C1]

(c) the reaction of the compound of formula (D1′) with a suitable fluorination agent in presence of a suitable solvent (iii) and optionally a suitable base (v), to arrive at a compound of formula (E1)

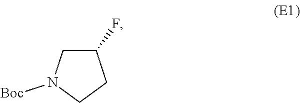 [SMILES: CC(C)(C)OC(=O)N1CC[C@@H](F)C1]

(d) the reaction of the compound of formula (E1) with a suitable acid (i) and a suitable solvent (iv), to arrive at a compound of formula (I) or an acceptable salt thereof.
3. A process for the preparation of a compound of formula (I),

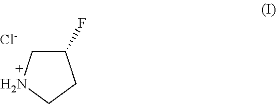

or an acceptable salt thereof, comprising the following steps:
(a) the reaction of a compound of formula (A1)

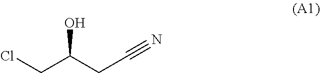

in presence of a suitable solvent (v) and in presence of H₂, a suitable catalyst, ammonia and ammonium chloride;
(b) adding a suitable base (iv) in a suitable solvent (vi) to arrive at the compound of formula (B1)

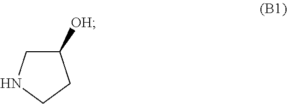

(c) optionally: (1) isolate the crude product of the compound of formula (B1) from the reaction mixture by means of distillation; (2) dissolving the crude product of a compound of formula (B1) in a suitable solvent (vii); and (3) isolate the crude product of the compound of formula (B1)) from the reaction mixture by means of distillation;
(d) the reaction of a compound of formula (B1)

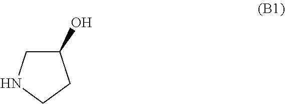

with a compound of formula (B2)

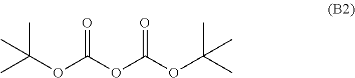

in presence of a suitable solvent (i), to arrive at a compound of formula (C1′)

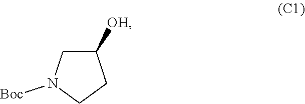 [SMILES: CC(C)(C)OC(=O)N1CC[C@H](O)C1]

(e) the reaction of the compound of formula (C1) with a compound of formula (C2′)

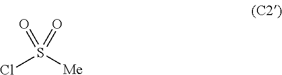

in presence of a suitable base (ii) and a suitable solvent (ii), to arrive at a compound of formula (D1′)

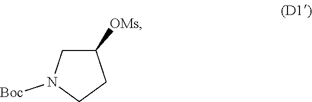 [SMILES: CC(C)(C)OC(=O)N1CC[C@H](OS(C)(=O)=O)C1]

(f) the reaction of the compound of formula (D1′) with a suitable fluorination agent in presence of a suitable solvent (iii) and a suitable base (v), to arrive at a compound of formula (E1)

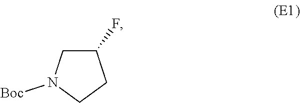 [SMILES: CC(C)(C)OC(=O)N1CC[C@@H](F)C1]

(g) the reaction of the compound of formula (E1) with a suitable acid (i) and a suitable solvent (iv), to arrive at a compound of formula (I) or an acceptable salt thereof.
4. A process according to any one of embodiment 1 to 3, further comprising the steps of
   (a′) dissolving the crude product of a compound of formula (I) in a suitable solvent (viii); and
   (b′) inducing crystallization by suitable means.
5. A process according to embodiment 4, wherein
   in step (a′), the solvent (viii) is a mixture of 1-butanol and water; and
   in step (b′), the crystallization is induced by azeotropic distillation of water.
6. A process for the preparation of a compound of formula (B1),

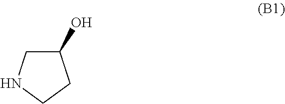

or an acceptable salt thereof, comprising the following steps:
(a) the reaction of a compound of formula (A1)

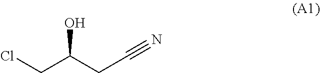

in presence of a suitable solvent (v) and in presence of H₂, a suitable catalyst, ammonia and ammonium chloride;
(b) adding a suitable base (iv) in a suitable solvent (vi) to arrive at the compound of formula (B1), or an acceptable salt thereof

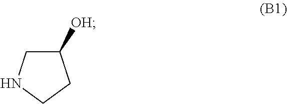

(c) isolate the crude product of the compound of formula (B1), or an acceptable salt thereof, from the reaction mixture by means of distillation;
and optionally further comprising the following steps:
(d) dissolving the crude product of a compound of formula (B1), or an acceptable salt thereof, in a suitable solvent (vii); and
(e) isolate the crude product of the compound of formula (B1)), or an acceptable salt thereof, from the reaction mixture by means of distillation.
7. A process according to any one of embodiments 3 to 6, wherein the reaction of step (a) was run as a pump hydrogenation.
8. A process according to any one of embodiments 3 to 6, wherein the reaction of step (a) was run as a batch process.
9. A process according to any one of embodiments 3 to 8, wherein
   in step (a) the catalyst is Raney-Cobalt, Raney-Nickel, Centoprime or Pd/C, in particular Raney-Cobalt.
10. A process according to any one of embodiments 3 to 9, wherein
   in step (b), the base (iv) is MeONa; and the solvent (vi) is MeOH.
11. A process according to any one of embodiments 3 to 10, wherein
   in step (c), distillation is initially performed at atmospheric pressure and then optionally at around 8-12 mbar.
12. A process according to any one of embodiments 3 to 8, wherein
   in step (a), the solvent (v) is methanol and the catalyst is Raney-Cobalt;
   in step (b), the base (iv) is MeONa; and the solvent (vi) is MeOH;
   in step (c), distillation is initially performed at atmospheric pressure and then optionally at around 8-12 mbar.
13. A process according to any one of embodiments 7, 9, 10, 11 and 12, wherein the reaction of step (a) is performed at between around 50° C. and around 110° C., in particular between around 60° C. and around 90° C., more particular between around 70° C. and around 80° C.; and wherein the H₂ pressure in step (a) is between around 10 bar and around 50 bar, particularly between around 10 bar and around 20 bar.
14. A process according to any one of embodiments 8 to 12, wherein the reaction of step (a) is performed at between around 50° C. and around 110° C., in particular between around 60° C. and around 90° C., more particular between around 70° C. and around 80° C.; and wherein the H₂ pressure in step (a) is between around 10 bar and around 50 bar, particularly between around 30 bar and around 50 bar.
15. A process according to any one of embodiments 4 to 14, wherein
   in step (c)(2), the solvent (vii) is a mixture of 1-propanol and hydrochloric acid; and
   in step (c)(3), distillation is initially performed at atmospheric pressure and then optionally at around 8-12 mbar.
16. A process according to any one of embodiments 1 to 5 or 7 to 15, wherein the fluorination agent is KF, CsF or TBAF, in particular KF.
17. A process according to any one of embodiments 1 to 5 or 7 to 16, wherein the solvent (iii) is diethylene glycol and the base (v) is NEt₃ or DIPEA, in particular NEt₃.
18. A process according to any one of embodiments 1 to 17, wherein the acceptable salt of the compound of formula (I) is an acceptable salt of the free base of the compound of formula (I) and wherein said salt is selected from mesylate, tosylate and camsylate, in particular mesylate.
19. A process for the preparation of a compound of formula (I),

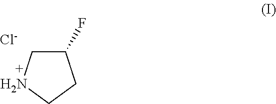

comprising the following steps:
(a) the reaction of a compound of formula (C1)

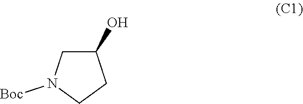 [SMILES: CC(C)(C)OC(=O)N1CC[C@H](O)C1]

with a compound (C2) selected from

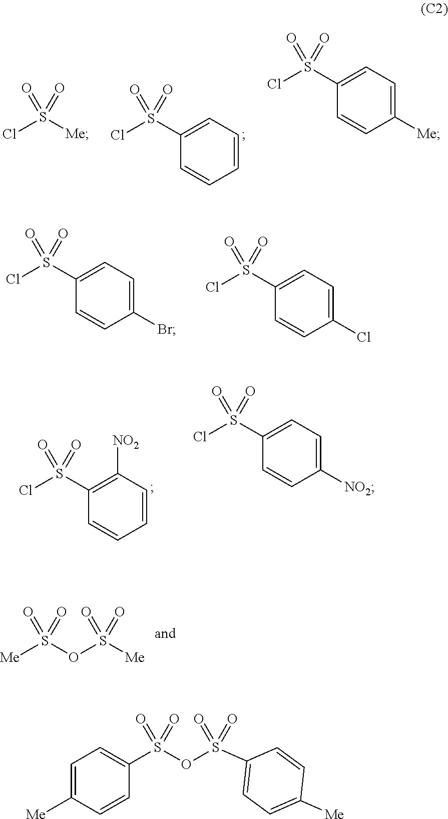

in presence of a suitable base (ii) and a suitable solvent (ii), to arrive at a compound of formula (D1)

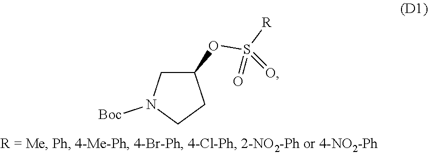

(b) the reaction of the compound of formula (D1) with a suitable fluorination agent in presence of a suitable solvent (iii) and optionally a suitable base (v), to arrive at a compound of formula (E1)

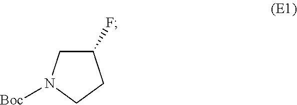 [SMILES: CC(C)(C)OC(=O)N1CC[C@@H](F)C1]

and
   (c) the reaction of the compound of formula (E1) with a suitable acid (i), in presence of a suitable solvent (iv), to arrive at a compound of formula (I).
20. A process according to any one of embodiments 1 to 19, wherein the compound (C2) is

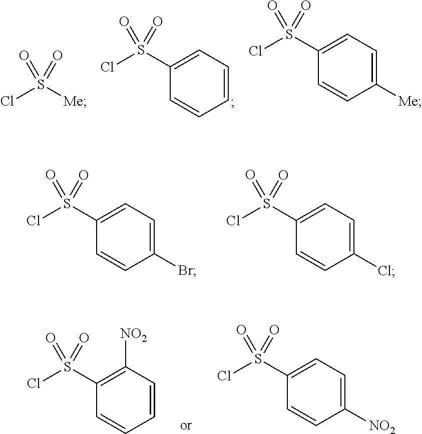

21. A process according to any one of embodiments 1 to 20, wherein compound (C2) is

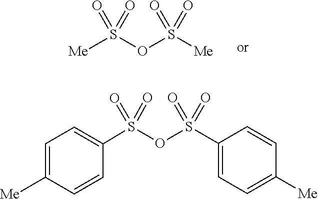

22. A process according to any one of embodiments 1 to 21, wherein compound (C2) is

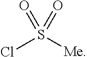

23. A process according to any one of embodiments 1 to 22, wherein the base (ii) is trimethylamine.
24. A process according to any one of embodiments 1 to 23, wherein the solvent (ii) is toluene or MTBE, in particular MTBE.
25. A process according to any one of embodiments 1 to 24, the solvent (iv) is n-BuOH, MTBE or a mixture thereof.
26. A process according to any one of embodiments 1 to 25, wherein the suitable acid (i) is HCl or methanesulfonic acid or HCl, in particular HCl.
27. A process according to any one of embodiments 1 to 26, wherein the solvent (i) is acetonitrile.
28. A process according to any one of embodiments 2 to 27, wherein step (a) of embodiment 2 comprises the reaction of a compound of formula (B1)

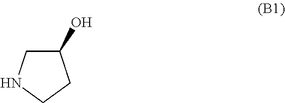

with a compound of formula (B2)

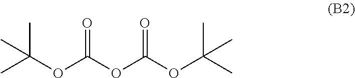

in presence of a suitable solvent (i) to arrive at a compound of formula (C1′)

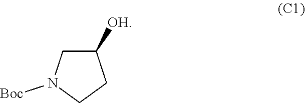 [SMILES: CC(C)(C)OC(=O)N1CC[C@H](O)C1]

29. A process according to any one of embodiments 2 to 27, wherein step (a) of embodiment 2 comprises the reaction of a compound of formula (B1′)

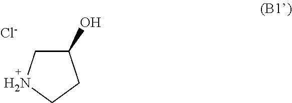

with a compound of formula (B2)

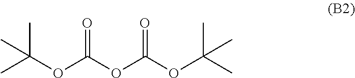

in presence of a suitable solvent (i) and a suitable base (i) to arrive at a compound of formula (C1′)

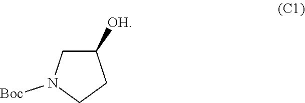 [SMILES: CC(C)(C)OC(=O)N1CC[C@H](O)C1]

30. A process according to any one of embodiments 2 to 27 or 29, wherein the base (i) is triethylamine.
31. A process according to any one of embodiments 1 to 30, wherein the solvent catalyst is Raney-Cobalt.
32. A process according to any one of embodiments 6 to 32, the pharmaceutically acceptable salt of the compound of formula (B1) is the compound of formula (B1′)

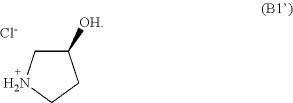

33. A process according to any one of embodiments 1 to 5 or 7 to 15, wherein the fluorination agent is KF, the solvent (iii) is diethylene glycol and the base (v) is NEt₃.
34. A compound selected from

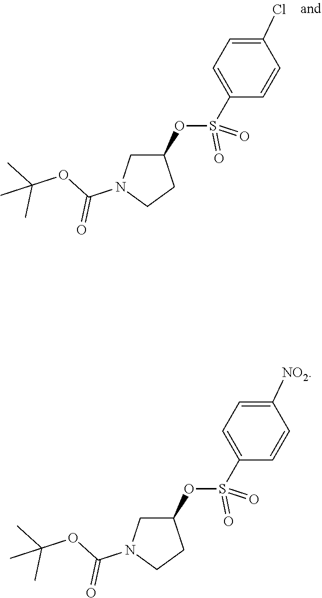 [SMILES: CC(C)(C)OC(=O)N1CC[C@H](OS(=O)(=O)c2ccc(Cl)cc2)C1 ; CC(C)(C)OC(=O)N1CC[C@H](OS(=O)(=O)c2ccc([N+](=O)[O-])cc2)C1]

The synthesis of the compound of formula (I) can, or example, be accomplished according to the non-exhaustive procedures described below in general scheme 1 or according to methods known to those skilled in the art.

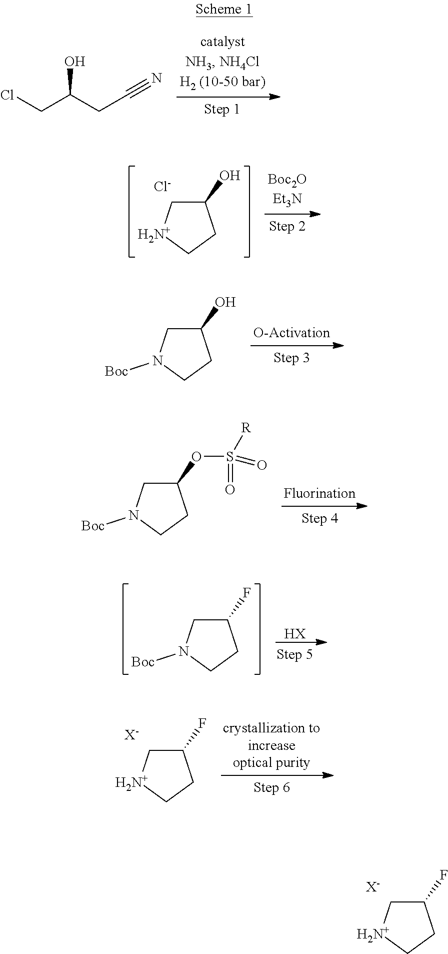

In the above scheme X can be for instance Cl or OMs, in particular Cl; and R can be for instance Me, Ph, 4-Me-Ph, 4-Br-Ph, 4-Cl-Ph, 2-NO₂-Ph or 4-NO₂-Ph, in particular Me.

In step 1, the catalyst can be for instance Raney-Cobalt, Pd/C or Centoprime, preferably Raney-Cobalt, in particular Raney-Cobalt. Ammonia and ammonium chloride are also present during the reaction of step 1. The solvent can be for instance MeOH, nBuOH or a mixture thereof.

In step 2, the solvent can be for instance acetonitrile or a mixture of MeOH, water and THF, in particular a mixture of MeOH, water and THF.

In step 3, the O-acetylation reagent can be for instance MsCl or 4-nosylate, the solvent can be for instance toluene, MTBE or a mixture thereof, and the base can be for instance triethylamine.

In step 4, the solvent can be for instance diethylene glycol and the fluorination agent can be for instance KF, CsF or TBAF, in particular KF.

In step 5, the acid can be for instance HCl or methanesulfonic acid, in particular HCl. In step 5, the solvent can be for instance n-BuOH, MTBE or a mixture thereof.

### EXPERIMENTAL PART

The following experiments are provided for illustration of the invention. They should not be considered as limiting the scope of the invention, but merely as being representative thereof. The steps and sub-steps described in Examples 1 to 2 can be combined to yield (3R)-fluoropyrrolidine, however the scope of the invention is not limited by the reactions specifically described in the examples but is to be construed in view of the whole disclosure of the present invention including inter alia also the general schemes and common general knowledge.

### Abbreviations

a/a=area/total area; ACN=acetonitrile; n-BuOH=1-butanol; DABCO=1,4-Diazabicyclo[2.2.2]octane; DIPEA=N,N-Diisopropylethylamine; EtOAc=ethyl acetate; GC=gas chromatography; HPLC=high-performance liquid chromatography; IPA=2-propanol; IPC=in process control; IT=inner temperature; LTL=loss to liquor; MCH=Methylcyclohexane; MeOH=methanol; MS=mass spectrometry; MsCl=mesyl chloride; MTBE=methyl tert-butyl ether; NEt₃=triethyl amine; NMR=nuclear magnetic resonance; NMT=not more than; SP=set point; TBAF=tetrabutylammonium fluoride; THF=tetrahydrofuran; V=volumes (litres per kilogram of limiting substrate).

### Example 1a

### tert-butyl (3S)-3-hydroxypyrrolidine-1-carboxylate (also referred to as N-Boc-(3S)-3-hydroxypyrrolidine)

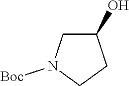 [SMILES: CC(C)(C)OC(=O)N1CC[C@H](O)C1]

### Step 1—Pump Hydrogenation:

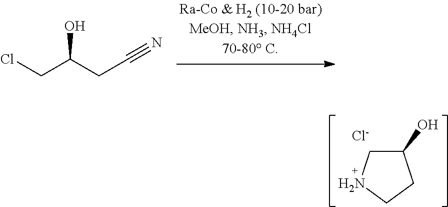

The catalyst Raney-Cobalt (12.4 wt-%; approx. 50% water-wet) is slurried in 0.7 V n-BuOH (volumes relative to (S)-4-chloro-3-hydroxybutyronitrile, technical bulk quality) a part of this amount can be withdrawn for rinsing) and transferred to a 1-liter autoclave (corrosion resistant, not stainless steel) under inert atmosphere (using nitrogen or argon). The stirrer is set to approx. 300 rpm. Subsequently, ammonium chloride (0.20 eq) is slurried in methanol (1.7 V, a part of this amount can be withdrawn for rinsing, technical bulk quality) and transferred to the autoclave. After addition of ammonia (1.05 eq. as 15% solution in MeOH) the autoclave is closed. The autoclave is rendered inert and the atmosphere in the reactor was exchanged to hydrogen. The suspension in the autoclave is heated to IT=70-80° C., then a hydrogen pressure of p=10-20 bar (aimed: 15 bar) is applied. (S)-4-Chloro-3-hydroxybutyronitrile (1.0 eq, 72.8 g) is dissolved in methanol (3.3 V, technical bulk quality) at room temperature. The starting material solution is transferred into an HPLC solvent flask (these steps can be prepared in advance) and attached to an HPLC pump. The content of the flask is pumped within at minimum 3 hours to the well stirred (1′000 rpm) suspension in the autoclave at IT=70-80° C. and a hydrogen pressure of p=10-20 bar. After end of dosage, the hydrogenation is allowed to resume for 60-90 minutes). The conversion of the starting material is checked by GC. Subsequently, the autoclave is cooled to IT=50-60° C. and the hydrogenation solution is filtered to remove the catalyst. The resulting hydrogenation solution exhibits a colorless to slightly yellow appearance (approx. 7.8 V) with a purity (GC) of 88%-a/a (Assay of product: 8.2%-w/w (GC), yielding 40.0 g product, 75% yield, 6.6% volumetric yield). The autoclave and the filter were rinsed with water (0.5 V) before discharge of the used catalyst.

### Step 2—Boc-Protection:

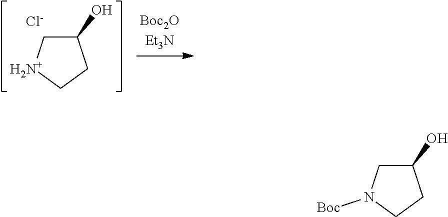

The hydrogenation solution, as obtained from step 1, was concentrated at 40-70° C. under reduced pressure to 2.5 V (volumes relative to (S)-4-chloro-3-hydroxybutyronitrile), Subsequently, 2.0 V of water were added and the mixture was concentrated at 40-70° C. under reduced pressure to 2.5 V. An additional 1.0 V of water were added at 20 to 25° C. and the pH was set to 10.8-11.2 with 30% NaOH aqueous (about 1.2 eq., about 1 V). The mixture was concentrated to 2.5 V under reduced pressure (jacket temperature: not more than 80° C.), followed by cooling to 20-25° C. and adjustment of the pH to 12.0 with 30% NaOH aqueous. Subsequently, the mixture was diluted with 2.0 V MeOH and Boc₂O (0.7 eq; as 70%-w/w solution in THF) was dosed at 25-35° C. over at least 2 hours. The reaction mixture was kept for 2 hours, followed by control of the conversion by GC (IPC: tert-butyl (3S)-3-hydroxypyrrolidine-1-carboxylate <1.0%). The reaction mixture was concentrated to 2.5 V at 40° C. to 70° C. under reduced pressure, followed by addition of 3.0 V MCH and 1 V water. The temperature was adjusted to 65° C. and the aqueous layer separated. Water was removed by azeotropic distillation (separation bottle) at normal pressure. Subsequently, 4%-w/w charcoal (CGP-Super) was added and the mixture was stirred for least 30 minutes at 50-65° C. The charcoal was filtered off and the filter cake rinsed with 1 V MCH. The solution was concentrated to 2.5 V (reduced pressure or normal pressure). 3%-w/w n-BuOH was added to the mixture and the temperature was adjusted to about 30° C. Subsequently, seeds of tert-butyl (3S)-3-hydroxypyrrolidine-1-carboxylate were added and the resulting mixture stirred for at least 30 minutes at about 30° C. The mixture was then cooled to about 0° C. over at least 1 hour and kept for 1 hour. The solids were filtered and the resulting cake washed with MCH. The solids were dried in a vacuum cabinet at about 30° C. to afford a white to off-white solid in 59.2% isolated yield with a purity (GC) of 99.89%-a/a.

### Example 1b (Analogue to Example 1a)

### Crude Reaction Mixture of (3S)-pyrrolidin-1-ium-3-ol hydrochloride (Also Referred to as (3S)-3-hydroxypyrrolidine hydrochloride)

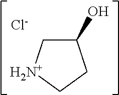

### Hydrogenation in Batch:

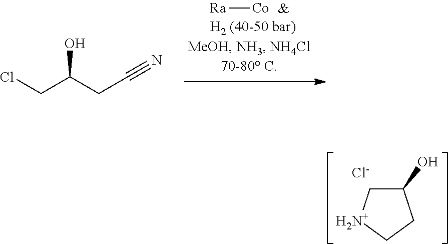

The catalyst Raney-Cobalt (12.4 wt-%; approx. 50% water-wet) is slurried in 0.7 V BuOH (volumes relative to (S)-4-chloro-3-hydroxybutyronitrile, a part of this amount can be withdrawn for rinsing) and transferred to a 1-liter autoclave (corrosion resistant, not stainless steel) under inert atmosphere (using nitrogen or argon). The stirrer is set to approx. 300 rpm. Subsequently, ammonium chloride (0.20 eq.) is slurried in methanol (1.7 V, a part of this amount can be withdrawn for rinsing) and transferred to the autoclave. Then, ammonia (1.05 eq. as 15% solution in MeOH) is added to the autoclave. (S)-4-Chloro-3-hydroxybutyronitrile (1.0 eq, 72.8 g) is dissolved in methanol (3.3 V) at room temperature and added to the autoclave. The autoclave is rendered inert and the atmosphere in the reactor is changed to hydrogen. The suspension in the autoclave is heated to IT=70-80° C., then a hydrogen pressure of p=40-50 bar (aimed: 45 bar) is applied. After end of hydrogen consumption, the hydrogenation is allowed to resume for 60-90 minutes (post hydrogenation). The conversion of the starting material is checked by GC. Subsequently, the autoclave is cooled to IT=50-60° C. and the hydrogenation solution is filtered to remove the catalyst. The resulting hydrogenation solution exhibits a yellow to brown appearance (approx. 7.8 V) with a purity (GC) of 68%-a/a (assay of product: 6.1%-w/w (GC), yielding 30.0 g product, 55% yield, 5.0% volumetric yield). The autoclave and the filter are rinsed with water (0.5 V) before discharge of the used catalyst.

### Example 1c

### (3S)-pyrrolidin-1-ium-3-ol hydrochloride (also referred to as (3S)-3-hydroxypyrrolidine hydrochloride)

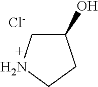

### Boc-Deprotection:

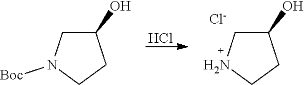 [SMILES: CC(C)(C)OC(=O)N1CC[C@H](O)C1 ; Cl ; [Cl-] ; O[C@H]1CCNC1]

N-Boc-(3S)-3-hydroxypyrrolidine (10 g, 1.0 eq.) was added to a mixture of IPA (10 ml, 1 V) and 5 M HCl in IPA (21.4 ml, 2.0 eq) at ambient temperature. The resulting reaction mixture stirred for 1 hour at 50° C. The slurry was cooled to 0° C. and filtered. The solids were dried in a vacuum cabinet at about 30° C. to afford the title compound in 84.5% isolated yield with a purity (GC) of 99.92%-a/a.

### Example 1d

### (3S)-pyrrolidin-1-ium-3-ol (Also Referred to as (3S)-3-hydroxypyrrolidine)

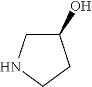

### Free Basing:

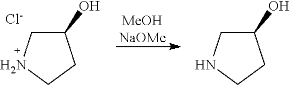 [SMILES: [Cl-] ; O[C@H]1CCNC1 ; "MeOHNaOMe" ; O[C@H]1CCNC1]

(3S)-3-hydroxypyrrolidine hydrochloride (149 kg, 1.0 eq, purity (GC)=99.78%-a/a) and MeOH (298 kg, 376 L, 2.5 V) were charged into a reactor at ambient temperature. Subsequently, a 30%-w/w solution of NaOMe in MeOH (217.1 kg, 1.0 eq) was added and the resulting mixture was stirred for 1 h at ambient temperature. The reaction mixture was filtered and the filter rinsed with MeOH (74.5 kg, 0.6 V). The combined filtrate was concentrated in vacuum at 50-55° C. until no liquid was distilled out. The residue was transferred to a high vacuum distillation reactor and distillation at 13-20 mbar and 105-115° C. afforded (3S)-3-hydroxypyrrolidine as a colorless oil in 92% isolated yield with a purity (GC) of 99.90%-a/a.

### Example 1e

### tert-butyl (3S)-3-hydroxypyrrolidine-1-carboxylate (Also Referred to as N-Boc-(3S)-3-hydroxypyrrolidine)

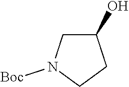 [SMILES: CC(C)(C)OC(=O)N1CC[C@H](O)C1]

### Boc-Protection:

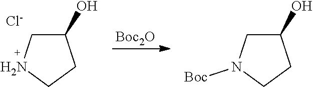 [SMILES: "Boc2O" ; [Cl-] ; O[C@H]1CCNC1 ; CC(C)(C)OC(=O)N1CC[C@H](O)C1]

(3S)-3-Hydroxypyrrolidine hydrochloride (100.0 g, 1.0 eq.) was added to a mixture of ACN (300 ml, 3 V) and triethylamine (98.3 g, 1.2 eq.) and the temperature was adjusted to 40-50° C. To this slurry, a solution of Boc₂O (185.4 g, 1.05 eq.) in ACN (100 ml, 1 V) was added over at least 1 hour. After the gas evolution had ceased, water (200 ml, 2 V) was added, and the biphasic mixture was concentrated until the internal temperature has reached 80-85° C. The residue was cooled to about 50-60° C., diluted with MTBE (300 ml, 3 V) and the aqueous layer drained. The aqueous layer was reextracted with MTBE (100 ml, 1 V) and the two organic layers were washed sequentially with water (100 ml, 1.0 V). The combined organic layers were concentrated to 250-300 ml at ambient pressure. The distillation was continued at constant volume while feeding MCH (500 ml, 5 V). The residue was diluted with 150 ml MCH, and the temperature adjusted to 30° C. The solution was seeded, stirred 1 h at 30° C., cooled to 0-5° C. over at least 1 hour and stirred at least 1 hour at this temperature. The product was isolated by filtration and the wet product washed with MCH (100 ml, 1 V). The product was dried in the cabinet at about 30° C. to afford N-Boc-(3S)-3-hydroxypyrrolidine in 94.4% isolated yield with a purity (GC) of 99.92%-a/a.

### Example 2a

### (3R)-3-fluoropyrrolidine hydrochloride

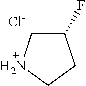

### Step 1—Mesylation:

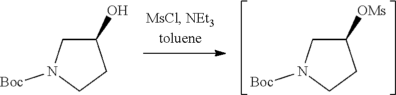

N-Boc-(3S)-3-hydroxypyrrolidine (125 g, 1.0 eq.) and NEt₃ (112 ml, 1.2 eq.) were dissolved in toluene (500 ml, 4 V) and the mixture cooled to 0° C. At this temperature, MsCl (80.3 g, 1.05 eq.) was added over at least 1 hour. Subsequently, the reaction temperature was raised to 20° C. The mixture was stirred for at least 30 min and the conversion was monitored by HPLC. The reaction mixture was quenched with deionised water (250 ml, 2 V). The aqueous layer was separated at 20° C. and the organic layer washed three times with/deionised water (3×125 ml, 3×1V). The organic phase was monitored for residual chloride (in case of residual amounts additional washes with deionized water can be applied).

### Step 2—Deoxylfuorination:

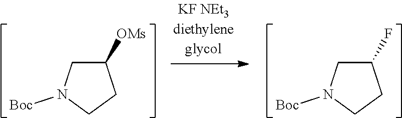

The organic layer from the previous step (mesylation) was concentrated to approx. 3 V (375 ml) at <60° C. under reduced pressure. Subsequently, KF (155.1 g, 4.0 eq) and diethylene glycol (750 ml, 6 V) were charged. The pressure was adjusted to 50 mbar and residual toluene was distilled off at increasing temperature. After the internal temperature has reached 80° C., the vacuum was broken with argon. Subsequently, NEt₃ (46.6 ml, 0.5 eq) was added and the reaction mixture was stirred at 80° C. for at least 16 hours, followed by an additional 5 hours at 95° C. The conversion was monitored by GC. The reaction mixture was diluted with water (250 ml, 2 V) and MTBE (250 ml, 2 V) and stirred for not less than 1 hour at 30-50° C. (SP: 45° C.). The lower water/diethylene glycol layer was drained and reextracted with MTBE (250 ml, 2 V). The MTBE layers were combined and washed twice with water (2×250 ml, 2×2V). The aqueous layers were monitored for residual fluoride (NMT 200 ppm; in case of residual amounts of fluoride the MTBE layer can be washed with additional amounts of dionized water). The solution of N-Boc-(3R)-3-fluoropyrrolidine in MTBE was unloaded and directly used in the next step.

### Step 3—Boc-Deprotection:

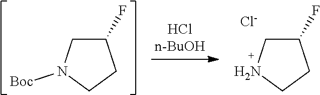

The solution of N-Boc-(3R)-3-fluoropyrrolidine in MTBE (about 640 ml) from the previous step, was placed in a glass reactor and distilled to 2.5 V at ambient pressure. Subsequently, n-BuOH (500 ml, 4 V) were added and the mixture was distilled to approx. 2 V (300 ml). The temperature was adjusted to 35° C. and HCl (20% w/w in n-BuOH) (152.1 g, 1.25 eq.) was added to the solution over at least 1 hour. The reaction mixture was stirred for at least 2 hours. Subsequently, the mixture was heated to 50° C. and stirred until the gas evolution has ceased (at least 2 hours). The resulting slurry was cooled to 20° C. over at least 1 hour, stirred 1 hour and the product isolated by filtration. The cake was washed with n-BuOH (50 ml, 0.4 V).

### Step 4—Recrystallization:

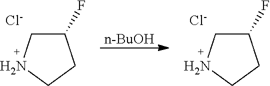 [SMILES: "n-BuOH" ; [Cl-] ; F[C@@H]1CCNC1 ; [Cl-] ; F[C@@H]1CCNC1]

The wet product is dissolved in a mixture of n-BuOH and water (315 mL, 2.5 V, V/V=95:5) at 60° C. Subsequently 1.50 V of the solvent is distilled off at 60-80° C. under reduced pressure (150 mbar), while keeping the volume constant by simultaneous feeding of n-BuOH (150 ml, 1.2 V). The resulting slurry was cooled to 20° C. over at least 1 hour, aged for 1 hour. The product was then filtered off. The cake was washed with n-BuOH (50 ml, 0.4 V). The wet product was subjected to another recrystallization under identical conditions and the resulting wet product was dried at 50° C. under vacuum. Pure (3R)-3-fluoropyrrolidine hydrochloride was obtained as an off-white solid in 52.4% (43.9 g) isolated yield with a purity of 99.89%-a/a and an optical purity of 99.98%-a/a.

### Example 2b

### tert-butyl (3R)-3-fluoropyrrolidine-1-carboxylate (Also Referred to as N-Boc-(3R)-3-fluoroypyrrolidine)

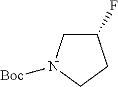 [SMILES: CC(C)(C)OC(=O)N1CC[C@@H](F)C1]

### Step 1—Mesylation:

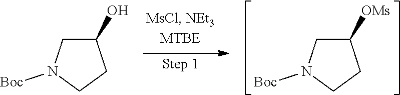

N-Boc-(3S)-3-hydroxypyrrolidine (100 g, 1.0 eq.) and NEt₃ (97 ml, 1.3 eq.) were dissolved in MTBE (400 ml, 4 V) and the mixture cooled to about −5-5° C. At this temperature, MsCl (64.2 g, 1.05 eq.) was added over at least 1 hour. The conversion was checked by TLC (toluene/EtOAc 1:1). The reaction mixture was quenched with deionized water (200 ml, 2 V). The aqueous layer was separated at 15-25° C. and the organic layer washed with deionized water (100 ml, 1 V). The organic layer was dried with MgSO₄ and evaporated to dryness to leave tert-butyl (3S)-3-methylsulfonyloxypyrrolidine-1-carboxylate as yellowish oil (quant. yield >99%).

### Step 2—Deoxyfluorination Using CsF:

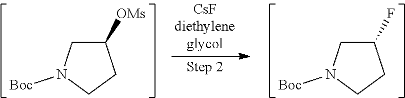

tert-butyl (3S)-3-methylsulfonyloxypyrrolidine-1-carboxylate (1.00 g, 1.00 eq.), CsF (2.29 g, 4.0 eq.) and diethylene glycol (6.0 ml, 6 V) were placed in a Pyrex tube and the mixture stirred at 80° C. for 18 hours. GC-analysis revealed the formation of 71.4% of the target N-Boc-(3R)-3-fluoroypyrrolidine (tert-butyl (3R)-3-fluoropyrrolidine-1-carboxylate).

### Example 2c

### tert-butyl (3R)-3-fluoropyrrolidine-1-carboxylate (Also Referred to as N-Boc-(3R)-3-fluoroypyrrolidine)

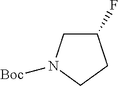 [SMILES: CC(C)(C)OC(=O)N1CC[C@@H](F)C1]

### Step 1—Mesylation:

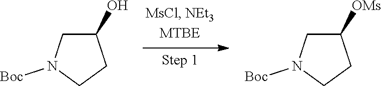 [SMILES: CC(C)(C)OC(=O)N1CC[C@H](O)C1 ; CC(C)(C)OC(=O)N1CC[C@H](OS(C)(=O)=O)C1]

N-Boc-(3S)-3-Hydroxypyrrolidine (100 g, 1.0 eq.) and NEt₃ (97 ml, 1.3 eq.) were dissolved in MTBE (400 ml, 4 V) and the mixture cooled to about −5-5° C. At this temperature, MsCl (64.2 g, 1.05 eq.) was added over at least 1 hour. The conversion was checked by TLC (toluene/EtOAc 1:1). The reaction mixture was quenched with deionized water (200 ml, 2 V). The aqueous layer was separated at 15-25° C. and the organic layer washed with deionized water (100 ml, 1 V). The organic layer was dried with MgSO₄ and evaporated to dryness to provide the tert-butyl (3S)-3-methylsulfonyloxypyrrolidine-1-carboxylate as yellowish oil (quant. yield >99%).

Step 2—Deoxylfuorination Using n-Bu₄NF (TBAF):

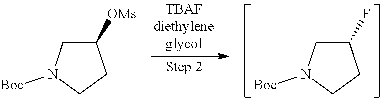

Tert-butyl (3S)-3-methylsulfonyloxypyrrolidine-1-carboxylate (1.00 g, 1.00 eq.), Bu₄NFx3H₂O (3.98 g, 4.0 eq.) and diethylene glycol (6.0 ml, 6 V) were placed in a Pyrex tube and the mixture stirred at about 80° C. for 18 hours. GC-analysis revealed the formation of 71.6% of the target tert-butyl (3R)-3-fluoropyrrolidine-1-carboxylate

### Example 2d

### (3R)-3-fluoropyrrolidine hydrochloride

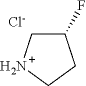

### Step 1—O-Activation:

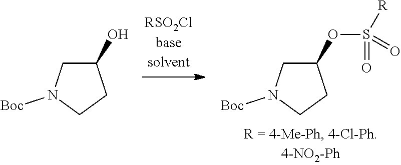

General procedure: To a mixture of N-Boc-(3S)-3-hydroxypyrrolidine (1.0 eq, optical purity >99.75%), base and additive in solvent was added the respective sulfonyl chloride (1.25 eq; see table below for details. The reaction mixture was aged for 1 hour at ambient temperature and the conversion checked by TLC. The reaction was quenched with water (5 V). The aqueous layer was separated, and the organic layer washed sequentially with 10% NaHSO₄ aqueous (5 V) and water (5 V). The organic layer was dried with MgSO₄ and evaporated to dryness. The residue was recrystallized from toluene/MCH and the products were isolated as a white solid.

| R | 4-Me—Ph | 4-Cl—Ph | 4-NO₂—Ph |
|---|---|---|---|
| Conditions | | | |
| O-activation reagent | 1.1 eq RSO₂Cl | 1.25 eq RSO₂Cl | 1.25 eq RSO₂Cl |
| base | 1.25 eq DABCO | 1.5 eq NEt₃ | 1.5 eq NEt₃ |
| additive | — | 0.2 eq DABCO | 0.2 eq DABCO |
| solvent | MTBE (10 V) | toluene (7 V) | toluene (7 V) |
| reaction temperature | ambient | ambient | ambient |
| Yield | 6.0 g | 19.3 g | 23.5 g |
| | 64% | 79% | 81% |
| Purity | | | |
| HPLC | n.d. | 99.6%-a/a | 100%-a/a |

### Step 2—Deoxyfluorination:

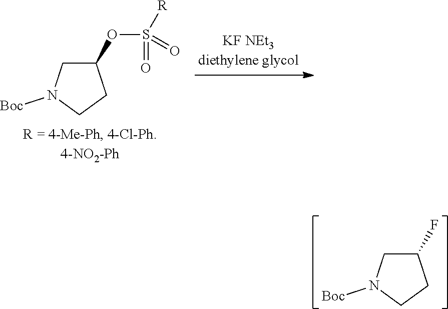

A mixture of the starting material and KF (4.0 eq.) in diethylene glycol (4 V) was stirred until complete conversion (time and temperature indicated in the table below). After an extractive workup the purity was analyzed by GC. To determine the chiral purity, samples of the crude product were deprotected with HCl in IPA, derivatized with FmocCl and analyzed by chiral HPLC.

| R | 4-Me—Ph | | 4-Cl—Ph | | 4-NO₂—Ph | |
|---|---|---|---|---|---|---|
| Fluorination | | | | | | |
| Scale | 1.8 | g | 15.0 | g | 20.0 | g |
| KF | 6.0 | eq | 5.0 | eq | 5.0 | eq |
| Diethylene glycol | 4 | V | 4 | V | 4 | V |
| Target temperature | 65° | C. | 65° | C. | 60° | C. |
| Reaction Time | 2 | days | 16 | h | 16 | h |
| Yield* | 440 | mg | 6.4 | g | 8.9 | g |

| | | | |
|---|---|---|---|
| | 66% | 82% | 88% |
| Purity | | | |
| GC | 69.4%-a/a | 85.0%-a/a | 84.2%-a/a |
| NMR | n.d. | 79.0% | 76.9% |
| Optical purity | | | |
| HPLC | 99.10%-a/a | 99.10%-a/a | 99.45%-a/a |

*crude product after extractive workup

### Step 3—Boc-Deprotection:

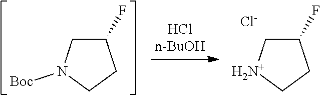

A solution of crude N-Boc-(3R)-3-fluoropyrrolidine in n-BuOH (1 V) from the previous step was added at 30-40° C. to a solution of HCl in n-BuOH (20%-w/w, 1.3 eq.) and stirred for 2 hours at this temperature. Then, the resulting slurry was cooled to 20° C. over at least 1 hour, stirred 1 hour and the product isolated by filtration. The cake was washed with n-BuOH (0.5 V) and dried under vacuum. No additional re-crystallization was performed. Chiral purity was determined by Fmoc derivatization with Fmoc-OSu and Chiralcel OZ-3R reverse phase column (ACN/H₂O; Na-Borate).

| R | 4-Me—Ph | 4-Cl—Ph | 4-NO₂—Ph |
|---|---|---|---|
| Purity | | | |
| GC | n.d. | n.d. | n.d. |
| NMR | n.d. | n.d. | n.d. |
| Optical Purity | | | |
| HPLC | 99.85%-a/a | 99.80%-a/a | 99.90%-a/a |
| Yield | 19.6 g | 2.6 g | 3.9 g |
| | 58.4% * | 50.0%** | 57.5%** |

* Starting from N-Boc-(3S)-3-hydroxypyrrolidine
**Starting from the corresponding O-activated N-Boc-(3S)-3-hydroxypyrrolidine

1. A process for the preparation of a compound of formula (I), 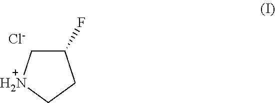
or an acceptable salt thereof, comprising the following steps:
(a) the reaction of a compound of formula (C1) 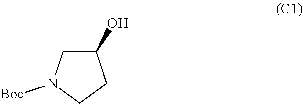 [SMILES: CC(C)(C)OC(=O)N1CC[C@H](O)C1]
with a compound (C2) selected from 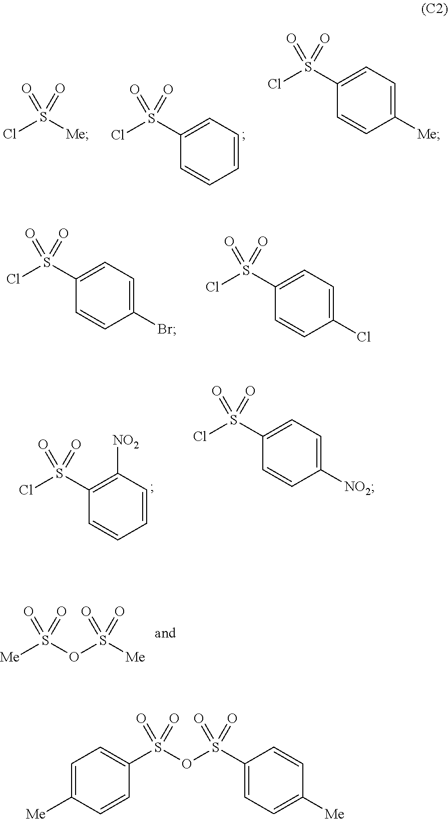
in presence of a suitable base (ii) and a suitable solvent (ii), to arrive at a compound of formula (D1) 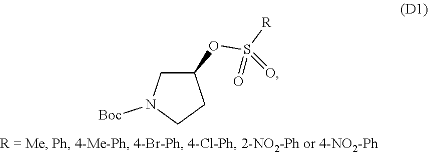
(b) the reaction of the compound of formula (D1) with a suitable fluorination agent in presence of a suitable solvent (iii) and optionally a suitable base (v), to arrive at a compound of formula (E1) 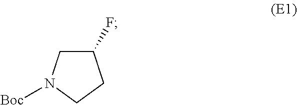 [SMILES: CC(C)(C)OC(=O)N1CC[C@@H](F)C1]
and
(c) the reaction of the compound of formula (E1) with a suitable acid (i), in presence of a suitable solvent (iv), to arrive at a compound of formula (I) or an acceptable salt thereof.

2. A process for the preparation of a compound of formula (I), 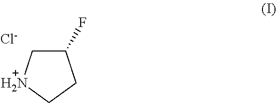
or an acceptable salt thereof, comprising the following steps:
(a) the reaction of a compound of formula (B1) or (B1′) 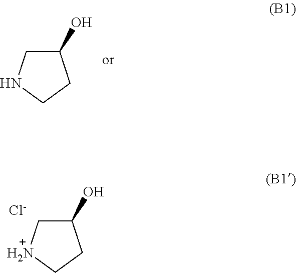 [SMILES: O[C@H]1CCNC1 ; [Cl-] ; O[C@H]1CC[NH2+]C1]
with a compound of formula (B2) 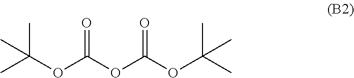
in presence of a suitable solvent (i) and in presence of a suitable base (i), to arrive at a compound of formula (C1′) 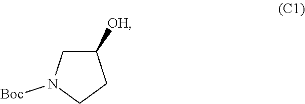 [SMILES: CC(C)(C)OC(=O)N1CC[C@H](O)C1]
(b) the reaction of the compound of formula (C1) with a compound of formula (C2′) 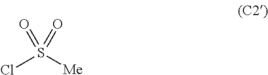
in presence of a suitable base (ii) and a suitable solvent (ii), to arrive at a compound of formula (D1′) 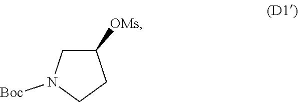 [SMILES: CC(C)(C)OC(=O)N1CC[C@H](OS(C)(=O)=O)C1]
(c) the reaction of the compound of formula (D1′) with a suitable fluorination agent in presence of a suitable solvent (iii) and optionally a suitable base (v), to arrive at a compound of formula (E1) 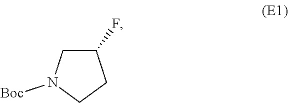 [SMILES: CC(C)(C)OC(=O)N1CC[C@@H](F)C1]
(d) the reaction of the compound of formula (E1) with a suitable acid (i) and a suitable solvent (iv), to arrive at a compound of formula (I) or an acceptable salt thereof.

3. A process for the preparation of a compound of formula (I), 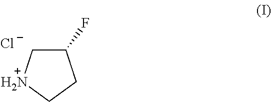
or an acceptable salt thereof, comprising the following steps:
(a) the reaction of a compound of formula (A1) 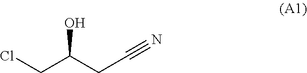
in presence of a suitable solvent (v) and in presence of H₂, a suitable catalyst, ammonia and ammonium chloride;
(b) adding a suitable base (iv) in a suitable solvent (vi) to arrive at the compound of formula (B1) 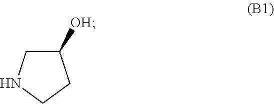
(c) optionally: (1) isolate the crude product of the compound of formula (B1) from the reaction mixture by means of distillation; (2) dissolving the crude product of a compound of formula (B1) in a suitable solvent (vii); and (3) isolate the crude product of the compound of formula (B1)) from the reaction mixture by means of distillation;
(d) the reaction of a compound of formula (B1) 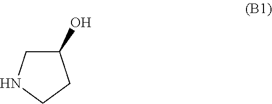
with a compound of formula (B2) 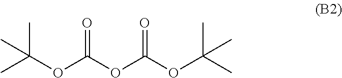
in presence of a suitable solvent (i), to arrive at a compound of formula (C1′) 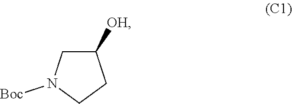 [SMILES: CC(C)(C)OC(=O)N1CC[C@H](O)C1]
(e) the reaction of the compound of formula (C1) with a compound of formula (C2′) 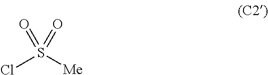
in presence of a suitable base (ii) and a suitable solvent (ii), to arrive at a compound of formula (D1′) 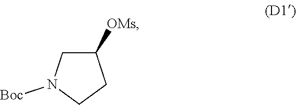 [SMILES: CC(C)(C)OC(=O)N1CC[C@H](OS(C)(=O)=O)C1]
(f) the reaction of the compound of formula (D1′) with a suitable fluorination agent in presence of a suitable solvent (iii) and a suitable base (v), to arrive at a compound of formula (E1) 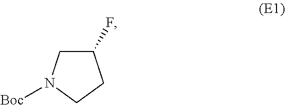 [SMILES: CC(C)(C)OC(=O)N1CC[C@@H](F)C1]
(g) the reaction of the compound of formula (E1) with a suitable acid (i) and a suitable solvent (iv), to arrive at a compound of formula (I) or an acceptable salt thereof.

4. The process according to claim 1, further comprising the steps of
(a′) dissolving the crude product of a compound of formula (I) in a suitable solvent (viii); and
(b′) inducing crystallization by suitable means.

5. The process according to claim 4, wherein
in step (a′), the solvent (viii) is a mixture of 1-butanol and water; and
in step (b′), the crystallization is induced by azeotropic distillation of water.

6. A process for the preparation of a compound of formula (B1), 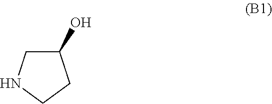
or an acceptable salt thereof, comprising the following steps:
(a) the reaction of a compound of formula (A1) 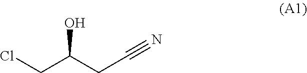
in presence of a suitable solvent (v) and in presence of H₂, a suitable catalyst, ammonia and ammonium chloride;
(b) adding a suitable base (iv) in a suitable solvent (vi) to arrive at the compound of formula (B1), or an acceptable salt thereof 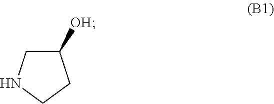
(c) isolate the crude product of the compound of formula (B1), or an acceptable salt thereof, from the reaction mixture by means of distillation;
and optionally further comprising the following steps:
(d) dissolving the crude product of a compound of formula (B1), or an acceptable salt thereof, in a suitable solvent (vii); and
(e) isolate the crude product of the compound of formula (B1)), or an acceptable salt thereof, from the reaction mixture by means of distillation.

7. The process according to claim 3, wherein the reaction of step (a) was run as a pump hydrogenation.

8. The process according to claim 3, wherein the reaction of step (a) was run as a batch process.

9. The process according to claim 3, wherein
in step (a) the catalyst is Raney-Cobalt, Raney-Nickel, Centoprime or Pd/C, in particular Raney-Cobalt.

10. The process according to claim 3, wherein
in step (b), the base (iv) is MeONa; and the solvent (vi) is MeOH.

11. The process according to claim 3, wherein
in step (c), distillation is initially performed at atmospheric pressure and then optionally at around 8-12 mbar.

12. The process according to claim 3, wherein
in step (a), the solvent (v) is methanol and the catalyst is Raney-Cobalt;
in step (b), the base (iv) is MeONa; and the solvent (vi) is MeOH;
in step (c), distillation is initially performed at atmospheric pressure and then optionally at around 8-12 mbar.

13. The process according to claim 7, wherein the reaction of step (a) is performed at between around 50° C. and around 110° C., in particular between around 60° C. and around 90° C., more particular between around 70° C. and around 80° C.; and wherein the H₂ pressure in step (a) is between around 10 bar and around 50 bar, particularly between around 10 bar and around bar.

14. The process according to claim 8, wherein the reaction of step (a) is performed at between around 50° C. and around 110° C., in particular between around 60° C. and around 90° C., more particular between around 70° C. and around 80° C.; and wherein the H₂ pressure in step (a) is between around 10 bar and around 50 bar, particularly between around 30 bar and around 50 bar.

15. The process according to claim 4, wherein
in step (c)(2), the solvent (vii) is a mixture of 1-propanol and hydrochloric acid; and
in step (c)(3), distillation is initially performed at atmospheric pressure and then optionally at around 8-12 mbar.

16. The process according to claim 1, wherein the fluorination agent is KF, NEt₃*3HF, CsF or TBAF, in particular KF.

17. The process according to claim 1, wherein the solvent (iii) is diethylene glycol and the base (v) is NEt₃ or DIPEA, in particular NEt₃.

18. The process according to claim 1, wherein the fluorination agent is KF, the solvent (iii) is diethylene glycol and the base (v) is NEt₃.

19. (canceled)
